SEQUENCE LISTING (4 sequences)

SEQ ID NO: 1 - DNA, 15 bp, synthetic construct
  source 1..15: mol_type = other DNA
tacagactttggaat

SEQ ID NO: 2 - DNA, 15 bp, synthetic construct
  source 1..15: mol_type = other DNA
atgactgaaacctta

SEQ ID NO: 3 - DNA, 18 bp, synthetic construct
  source 1..18: mol_type = other DNA
tacagactagtttggaat

SEQ ID NO: 4 - DNA, 18 bp, synthetic construct
  source 1..18: mol_type = other DNA
atgactgatcaaacctta

(19) United States
(12) Patent Application Publication
(10) Pub. No.: US 2026/0188428 A1
(43) Pub. Date: Jul. 2, 2026

(54) METHOD FOR DIAGNOSING AND PREDICTING CANCER TYPE USING FRAGMENT END MOTIF FREQUENCY AND SIZE OF CELL-FREE NUCLEIC ACID

(71) Applicant: GC GENOME CORPORATION, Gyeonggi-do (KR)

(72) Inventors: Eun-Hae CHO, Gyeonggi-do (KR); Tae-Rim LEE, Gyeonggi-do (KR)

(21) Appl. No.: 19/130,837

(22) PCT Filed: Nov. 29, 2023
(86) PCT No.: PCT/KR2023/019488

§ 371 Date: May 16, 2025

(30) Foreign Application Priority Data
Nov. 29, 2022 (KR) 10-2022-0162987

(51) Int. Cl. G16B 30/10; C12Q 1/6874; G16B 40/20

(52) U.S. Cl. CPC G16B 30/10; C12Q 1/6874; G16B 40/20

(57) ABSTRACT

The present invention relates to a method for diagnosing cancer and predicting cancer type using cell-free nucleic acid fragment end motif frequency and size, and more specifically, to a method for diagnosing cancer and predicting cancer type in which a nucleic acid is extracted from a biological sample, the end motif frequency of a nucleic acid fragment and the size of the nucleic acid fragment are derived on the basis of reads aligned by obtaining sequence information, and then a value calculated by generating the derived end motif frequency and size as vectorized data and post-processing same and then inputting the post-processed vectorized data to a learned artificial intelligence model is analyzed. A method for diagnosing cancer and predicting cancer type using the end motif frequency and size of a cell-free nucleic acid fragment according to the present invention generates vectorized data and analyzes same using an AI algorithm, and thus exhibits high sensitivity and accuracy even if read coverage is low. Therefore, the method is useful.

### TECHNICAL FIELD

The present invention relates to a method for diagnosing cancer and predicting a cancer type using fragment end motif frequencies and sizes of cell-free nucleic acid, and more preferably, to a method for diagnosing cancer and predicting a cancer type by extracting nucleic acids from a biological sample to obtain sequence information (reads), acquiring end motif frequencies and sizes of nucleic acid fragments based on the aligned reads, converting the end motif frequencies and sizes of nucleic acid fragments into vectorized data, post-processing the data, inputting the vectorized data to a trained artificial intelligence model and analyzing a resulting calculated value.

### BACKGROUND ART

Cancer diagnosis in clinical practice is usually performed by tissue biopsy after history examination, physical examination, and clinical evaluation. Cancer diagnosis based on clinical trials is possible only when the number of cancer cells is 1 billion or more and the diameter of the cancer is 1 cm or more. In this case, cancer cells already have the potential to metastasize and at least half thereof have already metastasized. In addition, tissue biopsy is invasive, which disadvantageously causes patients considerable discomfort and is often incompatible with cancer therapy. Further, tumor markers for monitoring substances produced directly or indirectly from cancer are used in cancer screening. However, the tumor markers have limited accuracy because more than half of tumor marker screening results indicate normal even in the presence of cancer and tumor marker screening results often indicate positive even in the absence of cancer.

Recently, in response to the requirements for cancer diagnosis methods, such as relative ease, non-invasiveness, high sensitivity and high specificity, liquid biopsy using bodily fluids from patients has been widely used for cancer diagnosis and follow-up examination. Liquid biopsy is a non-invasive diagnostic method that is attracting great attention as an alternative to conventional invasive diagnosis and examination methods.

Recently, a method for diagnosing cancer and determining a cancer type using cell free DNA obtained from liquid biopsy has been developed (U.S. patent Ser. No. 10/975,431, Zhou, Xionghui et al., bioRxiv, 2020.07.16.201350). In particular, a method of analyzing the motif frequency information of the cell-free nucleic acid end sequence and using the information for cancer diagnosis, prenatal diagnosis, or organ transplant monitoring is known (WO 2020-125709, Peiyong Jiang et al., Cancer Discovery, Vol. 10, 2020, pp. 664-673).

In addition, a method for diagnosing cancer using the ends of cell-free nucleic acids is known (US 2020-0199656 A1), but this method has the disadvantage of low accuracy.

Meanwhile, artificial neural networks are computational models implemented in software or hardware that mimic the computational ability of biological systems using a large number of artificial neurons connected via connective lines. Artificial neural networks use artificial neurons, which represent the functions of biological neurons in simplified form. Artificial neural networks conduct human cognition or learning processes by interconnecting the artificial neurons through connective lines having respective connection intensities. The term “connection intensity”, which is interchangeable with “connection weight”, refers to a predetermined value of the connection line. Artificial neural network learning may be classified into supervised learning and unsupervised learning. Supervised learning is a method of providing input data and output data corresponding thereto to a neural network and updating the connection intensities of connecting lines so that output data corresponding to the input data is output. Representative learning algorithms include delta rule and back propagation learning. Unsupervised learning is a method in which an artificial neural network independently learns connection intensities using only input data, without a target value. Unsupervised learning updates connection weights based on correlations between input patterns.

Applying large amounts of data to machine learning causes the so-called “curse of dimensionality” problem due to the increased complexity and the greater number of dimensions. In other words, as the number of dimensions of the required data approaches infinity, the distance between any two points also approaches infinity, and the amount of data, that is, the density, becomes lower in high-dimensional space, which makes it impossible to properly reflect the features of the data (Richard Bellman, Dynamic Programming, 2003, chapter 1). Recently developed deep learning has a structure in which a hidden layer is present between an input layer and an output layer, and has been reported to greatly improve the performance of the classifier in high-dimensional data such as images, videos, and signal data by processing a linear combination of variable values transmitted from the input layer with nonlinear functions (Hinton, Geoffrey, et al., IEEE Signal Processing Magazine Vol. 29.6, pp. 82-97, 2012).

Various patents (KR 10-2018-124550, KR 10-2019-7038076, KR 10-2019-0003676, and KR 10-2019-0001741) describe the use of artificial neural networks in biological fields, but there is a lack of research on methods for predicting cancer types through artificial neural network analysis based on cell-free DNA (cfDNA) sequencing information in blood.

Accordingly, as a result of extensive and earnest efforts to solve the above problems and develop a method for diagnosing cancer and predicting a cancer type based on artificial intelligence with high sensitivity and accuracy, the present inventors found that cancer diagnosis and cancer type prediction can be realized with high sensitivity and accuracy by generating vectorized data based on information on the end motifs and lengths of cell-free nucleic acid fragments and analyzing the data using a trained artificial intelligence model, and the present invention has been completed based on this finding.

### DISCLOSURE

Therefore, it is one object of the present invention to provide a method for diagnosing cancer and predicting a cancer type using end motif frequencies and sizes of cell-free nucleic acid fragments.

It is another object of the present invention to provide a device for diagnosing cancer and predicting a cancer type using the end motif frequencies and sizes of cell-free nucleic acid fragments.

It is another object of the present invention to provide a computer-readable storage medium including instructions configured to be executed by a processor for diagnosing cancer and predicting a cancer type by the method described above.

In accordance with one aspect of the present invention, provided is a method for providing information for diagnosing cancer and predicting a cancer type, the method including (a) extracting nucleic acids from a biological sample to obtain sequence information, (b) aligning the sequence information (reads) with a reference genome database, (c) acquiring end motif frequencies and sizes of nucleic acid fragments based on the aligned sequence information (reads), (d) generating vectorized data using the motif frequencies and sizes of nucleic acid fragments, (e) post-processing the vectorized data, (f) inputting the post-processed vectorized data into a trained artificial intelligence model, analyzing the data, and comparing an analyzed output value with a cut-off value to determine whether or not cancer develops, and (g) predicting a cancer type through comparison of the output value.

In accordance with one aspect of the present invention, provided is a method for diagnosing cancer and predicting a cancer type, the method including (a) extracting nucleic acids from a biological sample to obtain sequence information, (b) aligning the sequence information (reads) with a reference genome database, (c) acquiring end motif frequencies and sizes of nucleic acid fragments based on the aligned sequence information (reads), (d) generating vectorized data using the motif frequencies and sizes of nucleic acid fragments, (e) post-processing the vectorized data, (f) inputting the post-processed vectorized data into a trained artificial intelligence model, analyzing the data, and comparing an analyzed output value with a cut-off value to determine whether or not cancer develops, and (g) predicting a cancer type through comparison of the output value.

In accordance with another aspect of the present invention, provided is a device for diagnosing cancer and predicting a cancer type, the device including a decoder configured to extract nucleic acids from a biological sample and decode sequence information, an aligner configured to align the decoded sequences with a reference genome database, a nucleic acid fragment analyzer configured to acquire end motif frequencies and sizes of nucleic acid fragments based on the aligned sequences, a data generator configured to generate vectorized data using the end motif frequencies and sizes of nucleic acid fragments and then perform post-processing, a cancer diagnostic unit configured to input the post-processed vectorized data to a trained artificial intelligence model, analyze the data, compare a resulting output value with a cut-off value, and thereby determine whether or not cancer has developed, and a cancer type predictor configured to analyze the output value and thereby predict the cancer type.

In accordance with another aspect of the present invention, provided is a computer-readable storage medium for diagnosing cancer and predicting a cancer type including an instruction configured to be executed by a processor for diagnosing cancer and predicting a cancer type through the following steps including (a) extracting nucleic acids from a biological sample to obtain sequence information, (b) aligning the sequence information (reads) with a reference genome database, (c) acquiring end motif frequencies and sizes of nucleic acid fragments based on the aligned sequence information (reads), (d) generating vectorized data using the motif frequencies and sizes of nucleic acid fragments, (e) post-processing the generated vectorized data, (f) inputting the post-processed data to a trained artificial intelligence model, analyzing the data, and comparing an analyzed output value with a cut-off value to determine whether or not cancer develops, and (g) predicting a cancer type through comparison of the output value.

### DESCRIPTION OF DRAWINGS

FIG. 1 is an overall flowchart illustrating a method for diagnosing cancer and predicting a cancer type using end motifs and sizes of cell-free nucleic acid fragments according to the present invention;

FIG. 2 is an example of a process of selecting motifs having a difference in expression frequency between healthy subjects and cancer patients, or between respective cancer types according to an embodiment of the present invention;

FIG. 3 is a graph illustrating size distributions of nucleic acid fragments selected according to an embodiment of the present invention;

FIG. 4 illustrates an example in which an FEMS table is created from one nucleic acid fragment according to an embodiment of the present invention (left panel) and an example in which the FEMS table is created from all nucleic acid fragments;

FIG. 5 illustrates an example of a FEMS table created by further performing edge summary according to an embodiment of the present invention (left panel) and a result of visualization thereof (right panel);

FIG. 6 illustrates the difference in frequency between regions of the FEMS table produced according to one embodiment of the present invention.

FIG. 7 is a schematic diagram illustrating a process of producing an FEMS_Z table produced according to one embodiment of the present invention.

FIG. 8 is an example of visualization of the FEMS table produced based on data of healthy subjects and neuroblastoma patients used in one embodiment of the present invention and the FEMS_Z table constructed through standardization.

FIG. 9 shows the result of comparison in the performance of the CNN model using the FEMS table constructed according to one embodiment of the present invention and the CNN model using the FEMS_Z table.

FIG. 10 shows the result of an actual analysis on a patient in the CNN model using the FEMS table constructed according to one embodiment of the present invention and the CNN model using the FEMS_Z table.

FIG. 11 is a schematic diagram illustrating the configuration of the CNN model constructed according to one embodiment of the present invention.

### BEST MODE

Unless defined otherwise, all technical and scientific terms used herein have the same meanings as appreciated by those skilled in the field to which the present invention pertains. In general, the nomenclature used herein is well-known in the art and is ordinarily used.

Terms such as first, second, A, B, and the like may be used to describe various elements, but these elements are not limited by these terms and are merely used to distinguish one element from another. For example, without departing from the scope of the technology described below, a first element may be referred to as a second element and in a similar way, the second element may be referred to as a first element. “And/or” includes any combination of a plurality of related recited items or any one of a plurality of related recited items.

Singular forms are intended to include plural forms as well, unless the context clearly indicates otherwise. It will be further understood that the terms “comprises” and/or “comprising”, when used in this specification, specify the presence of features, numbers, steps, actions, components, parts, or combinations thereof, but do not preclude the presence or addition of one or more other features, numbers, steps, actions, components, parts, or combinations thereof.

Prior to the detailed description of the drawings, it is to be clarified that the classification of components in the present specification is merely made depending on the main function of each component. That is, two or more components described below may be combined into one component or one component may be divided into two or more depending on each more detailed function. In addition, each component to be described below may further perform some or all of the functions of other components in addition to its main function, and some of the main functions of each component may be performed exclusively by other components.

In addition, in implementing a method or operation method, respective steps constituting the method may occur in a different order from a specific order unless the specific order is clearly described in context. That is, the steps may be performed in the specific order, substantially simultaneously, or in reverse order to that specified.

It was found in the present invention that cancer diagnosis and cancer type prediction with high sensitivity and accuracy are possible by aligning sequencing data obtained from a sample with a reference genome, acquiring end motif frequencies and sizes of nucleic acid fragments based on the aligned sequence information (reads), generating vectorized data using the motif frequencies and sizes of nucleic acid fragments, post-processing the vectorized data, and calculating a DPI using a trained artificial intelligence model.

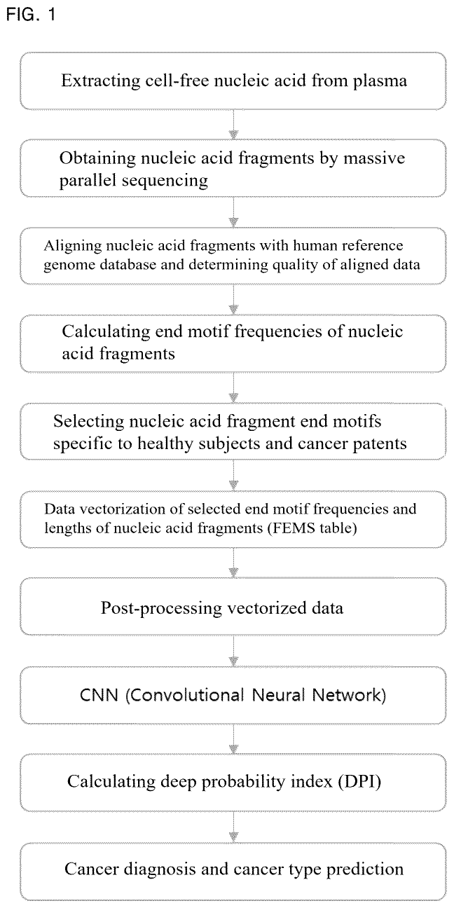

That is, in one embodiment of the present invention, developed is a method including sequencing DNA extracted from blood, aligning the sequencing data with a reference genome, acquiring end motif frequencies and sizes of nucleic acid fragments using the aligned sequence information, generating vectorized data with the end motif frequencies of nucleic acid fragments on the X-axis and the sizes of nucleic acid fragments on the Y-axis, post-processing the vectorized data, inputting the vectorized data to an artificial intelligence model trained to diagnose cancer and classify cancer types, and outputting the DPI, diagnosing cancer through comparison of the DPI with the cut-off value and then determining a type of cancer showing the highest DPI among the output DPIs for respective cancer types as the cancer type of the sample (FIG. 1).

In another aspect, the present invention is directed to a method for providing information for diagnosing cancer and predicting a cancer type, the method including:
(a) extracting nucleic acids from a biological sample to obtain sequence information;
(b) aligning the sequence information (reads) with a reference genome database;
(c) acquiring end motif frequencies and sizes of nucleic acid fragments based on the aligned sequence information (reads);
(d) generating vectorized data using the motif frequencies and sizes of nucleic acid fragments;
(e) post-processing the vectorized data;
(f) inputting the post-processed vectorized data into a trained artificial intelligence model, analyzing the data, and comparing an analyzed output value with a cut-off value to determine whether or not cancer develops; and
(g) predicting a cancer type through comparison of the output value.

In another aspect, the present invention is directed to a method for diagnosing cancer and predicting a cancer type, the method including:
(a) extracting nucleic acids from a biological sample to obtain sequence information;
(b) aligning the sequence information (reads) with a reference genome database;
(c) acquiring end motif frequencies and sizes of nucleic acid fragments based on the aligned sequence information (reads);
(d) generating vectorized data using the motif frequencies and sizes of nucleic acid fragments;
(e) post-processing the vectorized data;
(f) inputting the post-processed vectorized data into a trained artificial intelligence model, analyzing the data, and comparing an analyzed output value with a cut-off value to determine whether or not cancer develops; and
(g) predicting a cancer type through comparison of the output value.

In the present invention, any nucleic acid fragment can be used without limitation, as long as it is a fragment of a nucleic acid extracted from a biological sample, and the nucleic acid fragment is preferably a fragment of cell-free nucleic acid or intracellular nucleic acid, but is not limited thereto.

In the present invention, the nucleic acid fragment may be obtained by any method known to those skilled in the art, preferably direct sequencing, next-generation sequencing, sequencing through non-specific whole genome amplification, or probe-based sequencing, but the method is not limited thereto.

In the present invention, the cancer may be a solid cancer or a blood cancer, is preferably selected from the group consisting of non-Hodgkin lymphoma, Hodgkin lymphoma, acute-myeloid leukemia, acute-lymphoid leukemia, multiple myeloma, head and neck cancer, lung cancer, glioblastoma, neuroblastoma, colorectal/rectal cancer, pancreatic cancer, breast cancer, ovarian cancer, melanoma, prostate cancer, liver cancer, thyroid cancer, stomach cancer, gallbladder cancer, biliary tract cancer, bladder cancer, small intestine cancer, cervical cancer, cancer of unknown primary, kidney cancer, esophageal cancer and mesothelioma, and is more preferably neuroblastoma, but the cancer is not limited thereto.

In the present invention,
step (a) includes:
(a-i) obtaining nucleic acids from a biological sample;
(a-ii) removing proteins, fats, and other residues from the collected nucleic acids using a salting-out method, a column chromatography method, or a bead method to obtain purified nucleic acids;
(a-iii) producing a single-end sequencing or paired-end sequencing library for the purified nucleic acids or nucleic acids randomly fragmented by an enzymatic digestion, pulverization, or hydroshear method;
(a-iv) reacting the produced library with a next-generation sequencer; and
(a-v) obtaining sequence information (reads) of the nucleic acids in the next-generation sequencer.

In the present invention, the step (a) of obtaining sequence information may include obtaining the isolated cell-free DNA through whole genome sequencing at a depth of 1 million to 100 million reads.

In the present invention, the biological sample refers to any substance, biological fluid, tissue or cell obtained from or derived from a subject, and examples thereof include, but are not limited to, whole blood, leukocytes, peripheral blood mononuclear peripheral cells, leukocyte buffy coat, blood including plasma and serum, sputum, tears, mucus, nasal washes, nasal aspirates, breath, urine, semen, saliva, peritoneal washings, pelvic fluids, cyst fluids, meningeal fluid, amniotic fluid, glandular fluid, pancreatic fluid, lymph fluid, pleural fluid, nipple aspirate, bronchial aspirate, synovial fluid, joint aspirate, organ secretions, cells, cell extracts, semen, hair, saliva, urine, oral cells, placenta cells, cerebrospinal fluid, and mixtures thereof.

In the present invention, the next-generation sequencer may be used for any sequencing method known in the art. Sequencing of nucleic acids isolated using the selection method is typically performed using next-generation sequencing (NGS). Next-generation sequencing includes any sequencing method that determines the nucleotide sequence either of each nucleic acid molecule or of a proxy cloned from each nucleic acid molecule so as to be highly similar thereto (e.g., 105 or more molecules are sequenced simultaneously). In one embodiment, the relative abundance of nucleic acid species in the library can be estimated by counting the relative number of occurrences of the sequence homologous thereto in data produced by sequencing experimentation. Next-generation sequencing is known in the art, and is described, for example, in Metzker, M. (2010), Nature Biotechnology Reviews 11:31-46, which is incorporated herein by reference.

Platforms for next-generation sequencing include, but are not limited to, the FLX System genome sequencer (GS) from Roche/454, the Illumina/Solexa genome analyzer (GA), the Support Oligonucleotide Ligation Detection (SOLiD) system from Life/APG, the G. 007 system from Polonator, the HelioScope gene-sequencing system from Helicos Biosciences, and the PacBio RS system from Pacific Biosciences.

In the present invention, the length of the sequence information (reads) in step (b) may be 5 to 5,000 bp, and the number of sequence information (reads) that are used may be 5,000 to 5 million, but the present invention is not limited thereto.

In the present invention, the end motif of nucleic acid fragment in step (c) may be a sequence pattern of 2 to 30 bases at both ends of the nucleic acid fragment.

That is, with respect to a nucleic acid fragment sequenced by paired-end sequencing as shown below, the end motifs of the nucleic acid fragment are “TACA” sequentially read from the 5′ end of the forward strand and “ATTC” sequentially read from the 5′ end of the reverse strand.

Forward strand:
(SEQ ID NO: 1)
5′-TACAGACTTTGGAAT-3′
Reverse strand:
(SEQ ID NO: 2)
3′-ATGACTGAAACCTTA-5′

In the present invention, the frequency of the end motifs of the nucleic acid fragment in step (c) may be correspond to the number of motifs detected in all the nucleic acid fragments.

That is, when the end motif of the nucleic acid fragment is analyzed based on the four bases at both ends (4-mer motif), a combination of the four bases, namely, A, T, G, and C, located at the 1st, 2nd, 3rd, and 4th positions, respectively, is possible and thus motif values of a total of 256 (4*4*4*4) are analyzed.

The count of the number of motifs observed in the entire nucleic acid fragments produced by sequencing is referred to as “motif frequency” and the value calculated by dividing the motif frequency by the total number of nucleic acid fragments produced is referred to as “relative frequency”.

TABLE 1

| | AAAA | AAAC | AAAG | AAAT | AACA | AACC | . . . | TTTT | Row Sum |
|---|---|---|---|---|---|---|---|---|---|
| Forward | 62,639 | 105,142 | 127,299 | 75,485 | 399,505 | 42,583 | . . . | 269,530 | 63,319,687 |
| Strand | | | | | | | | | |
| Reverse | 62,432 | 105,719 | 126,493 | 75,788 | 400,900 | 42,467 | . . . | 269,802 | 63,110,437 |
| Strand | | | | | | | | | |
| Merged | 125,071 | 210,861 | 253,792 | 151,273 | 800,405 | 85,050 | . . . | 539,332 | 126,430,124 |
| End Motif | 0.00099 | 0.00167 | 0.00201 | 0.00120 | 0.00633 | 0.00067 | . . . | 0.00427 | — |
| Relative | | | | | | | | | |
| Freq | | | | | | | | | |

As shown in Table 1 above, the total number of nucleic acid fragments is 126, 430, 124, the number of nucleic acid fragments analyzed from “AAAA”, the end motif of the nucleic acid fragments is 125,071, the frequency of the end motif of the nucleic acid fragment, “AAAA”, is 125,071, and the relative frequency of end motifs of the nucleic acid fragments calculated by dividing the frequency by the total number of nucleic acid fragments is 0.00099.

In the present invention, the size of the nucleic acid fragment in step (c) may correspond to the number of bases from the 5′ end to the 3′ end of the nucleic acid fragment.

For example, the size of the nucleic acid fragment analyzed from SEQ ID NOs: 1 and 2 is 15.

In the present invention, the size of the nucleic acid fragment may be 1 to 10,000, preferably 10 to 1,000, more preferably 50 to 500, and most preferably 100 to 250, but the present invention is not limited thereto.

In the present invention, the vectorized data in step (d) may be expressed by the type of the end motif of the nucleic acid fragment plotted on the X-axis and the size of the nucleic acid fragment plotted on the Y-axis.

That is, assuming that there is one nucleic acid fragment as follows,

Forward strand:
(SEQ ID NO: 3)
5′-TACAGACTAGT . . . TTGGAAT-3′
Reverse strand:
(SEQ ID NO: 4)
3′-ATGACTGATCA . . . AACCTTA-5′

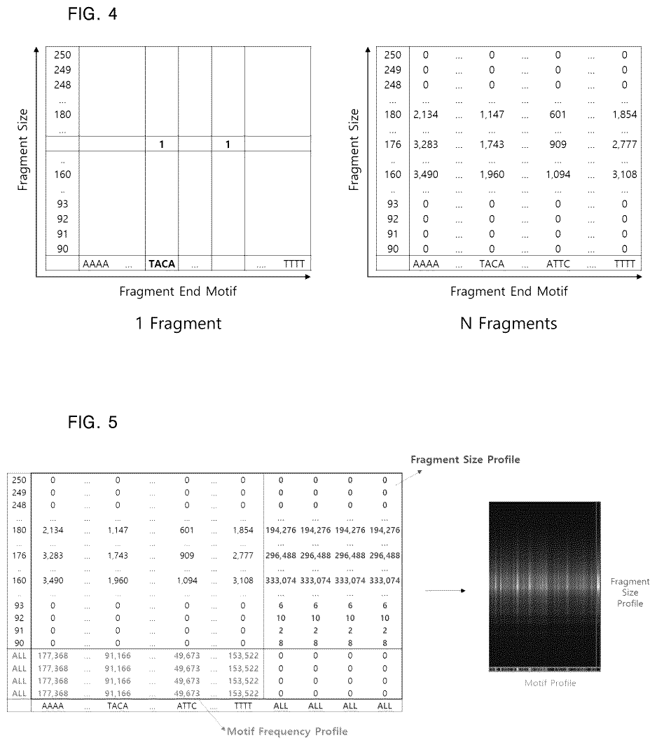

Fragment Size: 176
this nucleic acid fragment can be expressed as a two-dimensional vector as shown in the left panel of FIG. 4 and a two-dimensional vector as shown in the right panel of FIG. 4 is generated when this process is performed on an extended entire nucleic acid fragment and accumulated.

In the present invention, the vectorized data may further include the sum of the frequencies for end motifs of nucleic acid fragments and the sum of the frequencies for sizes of nucleic acid fragments.

That is, the two-dimensional vector as shown in the left panel of FIG. 5 is generated by further performing an edge summary by adding a column sum four times to the bottom of the two-dimensional vector in FIG. 4 in order to add frequency information for each fragment end motif irrelevant to the fragment size, and adding a row sum four times to the rightmost part of the two-dimensional vector of FIG. 4 in order to add the fragment size information irrelevant to the fragment end motif.

In the present invention, the two-dimensional vector is defined as a fragment end motif frequency and size (FEMS) table. The FEMS table is visualized and the result is shown in the right panel of FIG. 5.

In the present invention, step (e) may be performed by a method including the following steps:
(e-i) calculating the mean and standard deviation of the frequency of the end motifs of the nucleic acid fragment and size of the nucleic acid fragment in a group of normal subjects;
(e-ii) subtracting the mean of the frequency for the end sequence motif type of each nucleic acid fragment and the size of nucleic acid fragment in the normal group from the frequency for the end sequence motif type of each nucleic acid fragment and the size of nucleic acid fragment in the sample, and dividing the result by the standard deviation of the frequency for each type of motif and size of the nucleic acid fragment to perform Z standardization and thereby obtain a Z-standardized value; and
(e-iii) correcting the Z-normalized value derived in step (e-ii) with the cut-off value when the Z-normalized value exceeds a cut-off range.

In the present invention, the cut-off range may be −5 to 5 and the cut-off value may be −5 or 5, but is not limited thereto.

That is, the conventional FEMS table is characterized in that the difference in the distribution of calculated values for each area is great and post-processing for standardization of the difference is thus performed.

For example, the post-processing may be performed through the following steps:
i) selecting 99 healthy subjects included in the training data as a Z reference set;
ii) calculating means and standard deviations observed at each position in the FEMS table in the selected Z reference set, wherein, for example, the mean and standard deviation of values at the position (a) having a nucleic acid fragment size of 180 and having an AAAA motif were calculated in the FEMS table of the Z reference group of the 99 subjects, and defined as Mean 180_AAAA and SD 180_AAAA, respectively;
iii) performing Z standardization using the mean and standard deviation at each position in the FEMS table calculated in the (ii) process, wherein specifically, the frequency value observed at the position having a nucleic acid fragment size of 180 and the AAAA motif is defined as Value_180_AAAA, Z standardization was performed in accordance with the equation of Z 180_AAAA=(Value_180_AAAA−Mean_180_AAAA)/SD 180_AAAA; and
iv) limiting the minimum and maximum ranges of Z standardization values to −5 for Z<−5 and 5 for Z>5, in order to avoid the influence of Z standardization values that do not fall within the normal range (−5 to 5) due to the excessively small standard deviations.

The FEMS_Z table produced through the steps is visualized and the result is shown in FIG. 7.

In the present invention, the vectorized data is preferably an image, but is not limited thereto. An image is basically composed of pixels. If an image composed of pixels is vectorized, it may be expressed as a monochromatic 2D vector (black and white), a three-channel 2D vector (RGB colors), or a four-channel 2D vector (CMYK colors) depending on the type of image.

The vectorized data of the present invention is not limited to image data, and, for example, may be input data of an artificial intelligence model using an n-channel 2D vector (multi-channel vector) created by stacking n black-and-white images.

In the present invention, the vectorized data is preferably a 2D table, but is not limited thereto.

In the present invention, the method may further include, prior to step (c), separating nucleic acid fragments satisfying a mapping quality score from the aligned nucleic acid fragments.

In the present invention, the mapping quality score may vary depending on a desired criterion, but is preferably 15 to 70, more preferably 50 to 70, and most preferably 60.

In the present invention, any model may be used as the artificial intelligence model in step (f) without limitation, as long as it can be trained to distinguish between images of cancer types and the artificial intelligence model is preferably a deep-learning model.

In the present invention, the artificial intelligence model may be any artificial neural network algorithm capable of analyzing vectorized data based on an artificial neural network without limitation and is preferably selected from the group consisting of a convolutional neural network (CNN), a deep neural network (DNN), and a recurrent neural network (RNN), but is not limited thereto.

In the present invention, the recurrent neural network is selected from the group consisting of a long-short term memory (LSTM) neural network, a gated recurrent unit (GRU) neural network, a vanilla recurrent neural network, and an attentive recurrent neural network.

In the present invention, when the artificial intelligence model is a CNN, the loss function for performing binary classification is represented by Equation 1 below, and the loss function for performing multi-class classification is represented by Equation 2 below.

$$Binary classification$$

$$loss ( model ( x ) , y ) = - \frac{1}{n} [ {{\sum }_{i = 1}}^{n} ( {y}_{i} log ( model ( {x}_{i} ) ) + ( 1 - {y}_{i} ) log ( 1 - model ( {x}_{i} ) ) ) ] \qquad Equation 1$$

Model (xᵢ)=Artificial intelligence model output in response to i^{th} input
y=Actual label value
n=Number of input data
Equation 2: Multi-class classification

$$loss ( model ( x ) , y ) = - \frac{1}{n} {{\sum }_{i = 1}}^{n} ( {{\sum }_{j = 1}}^{c} ( {y}_{ij} {log ( model ( {x}_{i} ) )}_{j} )$$

Model (xᵢ); =j^{th} artificial intelligence model output in response to i^{th} input
y=Actual label value
n=Number of input data
c=Number of classes

In the present invention, the binary classification means that the artificial intelligence model learns to determine whether or not cancer develops, and multi-class classification means that the artificial intelligence model learns to distinguish between two or more cancer types.

In the present invention, when the artificial intelligence model is a CNN, learning includes the following steps:
i) classifying the generated vector data into training, validation, and test data,
wherein the training data is used when the CNN model is trained, the validation data is used for hyper-parameter tuning validation, and the test data is used for the test after optimal model production; and
ii) constructing an optimal CNN model through hyper-parameter tuning and training; and
iii) comparing the performance of multiple models obtained through hyper-parameter tuning using validation data and determining the model having the best validation data to be the optimal model.

In the present invention, hyper-parameter tuning is a process of optimizing the values of various parameters (the number of convolution layers, the number of dense layers, the number of convolution filters, etc.) constituting the CNN model. The hyper-parameter tuning is performed using Bayesian optimization and grid search methods.

In the present invention, the internal parameters (weights) of the CNN model are optimized using predetermined hyper-parameters, and it is determined that the model is over-fit when validation loss starts to increase compared to training loss and then training is stopped.

In the present invention, any value resulting from analysis of the input vectorized data by the artificial intelligence model in step (f) may be used without limitation, as long as it is a specific score or real number, and the value is preferably a deep probability index (DPI), but is not limited thereto.

As used herein, the term “deep probability index” refers to a value expressed as a probability value by adjusting the output of artificial intelligence to a scale of 0 to 1 using a sigmoid function in binary classification and a softmax function in multi-class classification for the last layer of the artificial intelligence model.

In binary classification, training is performed using the sigmoid function such that the DPI is adjusted to 1, provided that cancer develops. For example, when a breast cancer sample and a normal sample are input, training is performed such that the DPI of the breast cancer sample is close to 1.

In multi-class classification, as many DPIs as the number of classes are extracted using the softmax function. The sum of the DPIs is adjusted to 1 and training is performed such that the DPI of the cancer type is actually adjusted to 1. For example, provided that there are three classes, namely, breast cancer, liver cancer, and normal group, when a breast cancer sample is input, training is performed to adjust a DPI of the breast cancer class to about 1.

In the present invention, the resulting output value of step (f) is obtained for each cancer type.

In the present invention, the artificial intelligence model is trained to adjust an output value to about 1 if there is cancer and to adjust an output value to about 0 if there is no cancer. Therefore, performance (training, validation, test accuracy) is measured based on a cut-off value of 0.5. In other words, if the output value is 0.5 or more, it is determined that there is cancer, and if it is less than 0.5, it is determined that there is no cancer.

Here, it will be apparent to those skilled in the art that the cut-off value of 0.5 may be arbitrarily changed. For example, in an attempt to reduce false positives, the cut-off value may be set to be higher than 0.5 as a stricter criterion for determining whether or not there is cancer, and in an attempt to reduce false negatives, the cut-off value may be set to be lower than 0.5 as a weaker criterion for determining that there is cancer.

Most preferably, the cut-off value can be set by determining the probability of the DPI by applying unseen data (data containing a solution that is different from that trained during training) using the trained artificial intelligence model.

In the present invention, (g) predicting a cancer type through comparison of the output result includes determining the cancer type showing the highest value among the output result values as the cancer of the sample.

In another aspect, the present invention is directed to a device for diagnosing cancer and predicting a cancer type, the device including: a decoder configured to extract nucleic acids from a biological sample and decode sequence information;
an aligner configured to align the decoded sequence with a reference genome database;
a nucleic acid fragment analyzer configured to acquire end motif frequencies and sizes of nucleic acid fragments based on the sequence;
a data generator configured to generate vectorized data using the end motif frequencies and sizes of nucleic acid fragments and perform post-processing;
a cancer diagnostic unit configured to input the post-processed vectorized data to a trained artificial intelligence model, analyze the data, compare the resulting value with a cut-off value, and thereby determine whether or not cancer develops; and
a cancer type predictor configured to analyze the output value and thereby predict the cancer type.

In the present invention, the decoder may include a nucleic acid injector configured to inject the nucleic acid extracted from an independent device, and a sequence information analyzer configured to analyze the sequence information of the injected nucleic acid, preferably an NGS analyzer, but is not limited thereto.

In the present invention, the decoder may receive and decode sequence information data generated in an independent device.

In another aspect, the present invention is directed to a computer-readable storage medium for diagnosing cancer and predicting a cancer type including an instruction configured to be executed by a processor for diagnosing cancer and predicting a cancer type through the following steps including:
(a) extracting nucleic acids from a biological sample to obtain sequence information;
(b) aligning the sequence information (reads) with a reference genome database;
(c) acquiring end motif frequencies and sizes of nucleic acid fragments based on the aligned sequence information (reads);
(d) generating vectorized data using the motif frequencies and sizes of nucleic acid fragments;
(e) post-processing the generated vectorized data;
(f) inputting the post-processed data to a trained artificial intelligence model, analyzing the data, and comparing an analyzed output value with a cut-off value to determine whether or not cancer develops; and
(g) predicting a cancer type through comparison of the output value.

In another aspect, the method according to the present disclosure may be implemented using a computer. In one embodiment, the computer includes one or more processors coupled to a chipset. In addition, a memory, a storage device, a keyboard, a graphics adapter, a pointing device, a network adapter and the like are connected to the chipset. In one embodiment, the performance of the chipset is acquired by a memory controller hub and an I/O controller hub. In another embodiment, the memory may be directly coupled to a processor instead of the chipset. The storage device is any device capable of maintaining data, including a hard drive, compact disc read-only memory (CD-ROM), DVD, or other memory devices. The memory is concerned with data and instructions used by the processor. The pointing device may be a mouse, track ball or other type of pointing device, and is used in combination with a keyboard to transmit input data to a computer system. The graphics adapter presents images and other information on a display. The network adapter is connected to the computer system through a local area network or a long distance communication network. However, the computer used herein is not limited to the above configuration, may not have some configurations, may further include additional configurations, and may also be part of a storage area network (SAN), and the computer of the present invention may be configured to be suitable for the execution of modules in the program for the implementation of the method according to the present invention.

The module used herein may mean a functional and structural combination of hardware to implement the technical idea according to the present invention and software to drive the hardware. For example, it is apparent to those skilled in the art that the module may mean a logical unit of a predetermined code and a hardware resource to execute the predetermined code, and does not necessarily mean a physically connected code or one type of hardware.

### EXAMPLE

Hereinafter, the present invention will be described in more detail with reference to examples. However, it will be obvious to those skilled in the art that these examples are provided only for illustration of the present invention and should not be construed as limiting the scope of the present invention.

### Example 1. Extracting DNA from Blood to Perform Next-Generation Sequencing

10 mL of blood was collected from each of 202 normal subjects and 64 neuroblastoma cancer patients, and stored in an EDTA tube. Within 2 hours of blood collection, only the plasma was primarily centrifuged at 1,200 g and 4° C. for 15 minutes, and then the primarily centrifuged plasma was secondarily centrifuged at 16,000 g and 4° C. for 10 minutes to isolate the plasma supernatant excluding the precipitate. Cell-free DNA was extracted from the isolated plasma using a chemagic ccfNA 2K Kit (chemagen), a library preparation process was performed using a MGIEasy cell-free DNA library prep set kit, and then sequencing was performed in a 100 base paired end mode using a DNBseq G400 device (MGI). As a result, about 170 million reads were found to be produced from each sample.

The generated dataset is shown in Table 2 below.

TABLE 2

| Sample Type | Train | Validation | Test | Total |
|---|---|---|---|---|
| Normal | 99 | 42 | 61 | 202 |
| Cancer (NBT) | 30 | 13 | 18 | 61 |

### Example 2. Selection of Nucleic Acid Fragment End Motif and Nucleic Acid Fragment Size

### 2-1. Selection of Nucleic Acid Fragment End Motif

The nucleic acid fragment end motifs were determined from 4 bases (A, T, G, C), and among a total of 256 (4*4*4*4) motifs, some motifs had no relative frequency difference between normal and NBT groups. A FEMS table generated including a motif not having such a difference may act as noise that only increases the amount of computation of the model without providing information essential for classification. Therefore, in order to exclude these meaningless motifs, only specific motifs having significant relative frequency differences between the three groups were selected.

In addition, in order to prevent the model overfitting issue in the size and motif selection process, only the training set was used in the size and motif selection process.

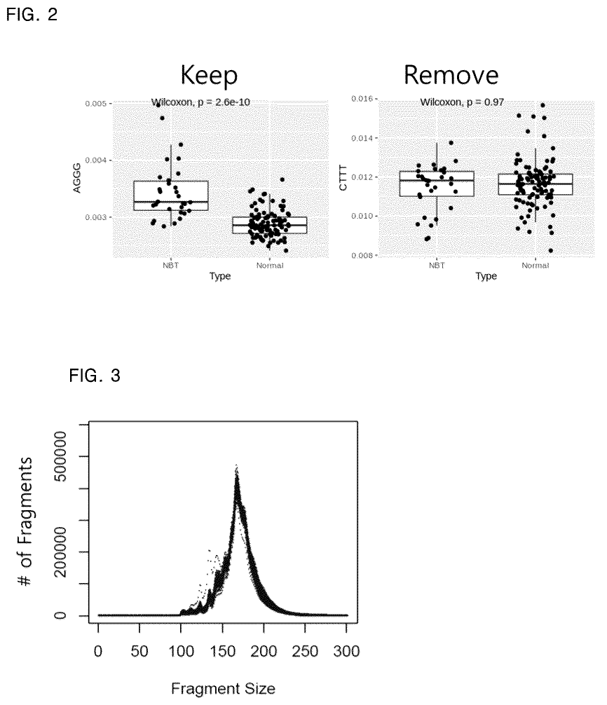

That is, the nucleic acid fragment end motifs were set with 4 bases (A, T, G, C) using the NGS data generated in Example 1 and some motifs that had statistically significant (Kruskal-Wallis Test, FDR-adjust p<0.05) relative frequency difference between healthy subjects (Normal) and neuroblastoma (NBT) patient groups were selected from a total of 256 types (4*4*4*4) of motifs (FIG. 2).

In addition, motifs having an average frequency higher than the random baseline ( 1/256, 0.004) in the healthy subject group were further selected from the motifs selected through the above process in order to prevent overfitting.

As a result, a total of 85 motifs was obtained and detailed motif information is as follows:

CACT, CCCC, CCAT, TATT, ACCA, AGCA, TACA, CCTC,
ACAA, TGTT, TGCT, CTCT, GGTA, GGCT, ATTT, TGTC,
GCCT, GACA, CACC, CATA, CACA, TACT, AGTA, TATC,
GGAG, TCTC, AGTG, TGTG, GGCA, GGGA, GCCA, CATC,
AATA, TGAT, TGAC, CTGA, GAAT, AACA, CATG, TGAA,
GCTG, CTTG, GGTG, GGAT, CAAG, TATG, GAAA, CTTC,
GGAA, AAAT

### 2-2. Nucleic Acid Fragment Size Selection

Most of the nucleic acid fragments whose quality has been checked have a size in the range of 110 to 230, as shown in FIG. 3. Therefore, when a FEMS table including an area that is out of this size range, most areas are filled with zero (0) and only meaningless noise increases. For this reason, the nucleic acid fragment size was selected within this range.

### Example 3. Production of Fragment End Motif Frequency and Size (FEMS) Table and Production of FEMS_Z Table

### 3-1 Production of FEMS Table

Two-dimensional vectors were generated by plotting motif types on the X-axis and fragment sizes on the Y-axis to simultaneously express the end motif frequency and size information of the nucleic acid fragments selected in Example 2. More specifically, as shown in the left panel of FIG. 4, the type and size of nucleic acid motifs at both ends of one nucleic acid fragment are expressed as a frequency, and this is extended to the entire nucleic acid fragment and accumulated, to generate two-dimensional vectors as shown in FIG. 4.

Also, edge summary was further performed by adding a column sum four times to the bottom of the two-dimensional vector in FIG. 4 in order to add frequency information for each fragment end motif irrelevant to the fragment size, and adding a row sum four times to the rightmost part of the two-dimensional vector of FIG. 4 in order to add the fragment size information irrelevant to the fragment end motif, to generate a two-dimensional vector as shown in the left panel of FIG. 5. The two-dimensional vector is defined as a fragment end motif frequency and size (FEMS) table. The FEMS table was visualized and an example thereof is shown in FIG. 5.

### 3-2 Production of FEMS_Z Table

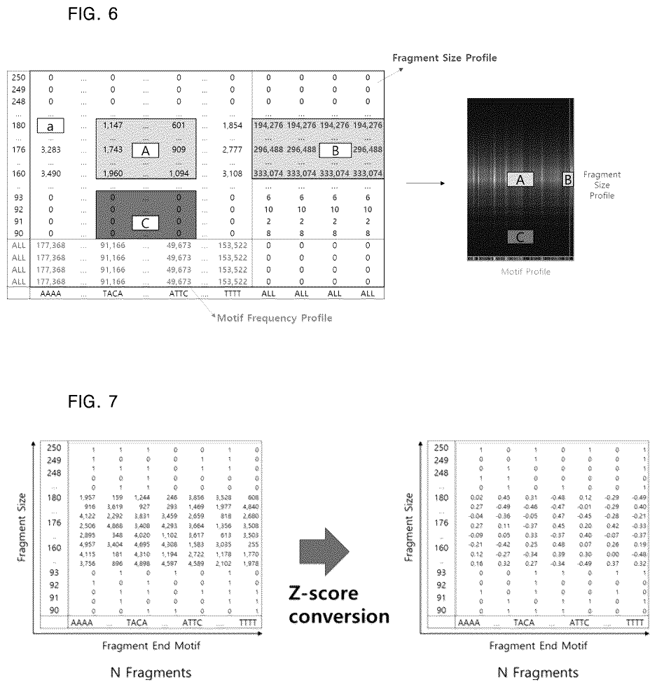

The values constituting the FEMS table formed in 3-1 mean the frequencies of nucleic acid fragments specific sizes and motifs. As shown in FIG. 6, this frequency value is characterized in that there is a large difference in the distribution between values calculated in relatively high-frequency regions (A and B) and low-frequency regions (C). For example, a difference of 100 units is observed in region A, a difference of 10,000 units is observed in region B, whereas a difference of only 1 unit is rarely observed in region C. When this FEMS table is used, there is a problem in that it becomes difficult for CNN-based AI algorithms to learn parameters (weights). Therefore, additional pretreatment was performed to have values in a similar range in all areas of the FEMS table to create the FEMS_Z table.

Specifically, 99 healthy subjects included in the training data in Table 2 were selected as a Z reference set and means and standard deviations observed at each position in the FEMS table in the selected Z reference set were calculated.

For example, the mean and standard deviation of values at the position (a) having a nucleic acid fragment size of 180 and having an AAAA motif were calculated in the FEMS table of the Z-reference group of 99 subjects, and defined as Mean_180_AAAA and SD 180_AAAA, respectively.

Z standardization was performed using the mean and standard deviation at each position in the FEMS table calculated in the above process. Specifically, the frequency value observed at the position having a nucleic acid fragment size of 180 and the AAAA motif is defined as Value_180_AAAA, Z standardization was performed in accordance with the equation of Z_180_AAAA=(Value_180_AAAA−Mean_180_AAAA)/SD_180_AAAA (FIG. 7).

In order to avoid the influence of Z standardization values that do not fall within the normal range (−5 to 5) due to the excessively small standard deviations, the minimum and maximum ranges of Z standardization values were limited to −5 for Z<−5 and 5 for Z>5.

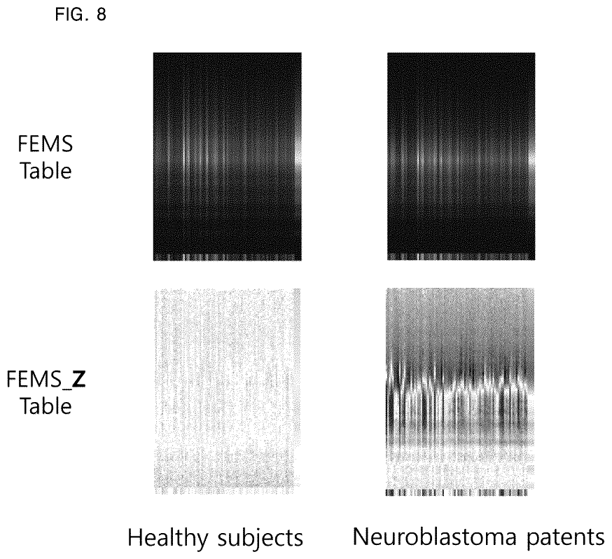

In the above process, 2D vectors obtained by substituting values of all positions in the conventional FEMS table with Z-standardized values were defined in the FEMS_Z table, and a visual comparison of the FEMS table with the FEMS_Z table is shown in FIG. 8.

The FEMX_Z table was formed using an edge summary including adding the column sum 4 times to the bottom of the 2D vector in order to add frequency information for each fragment end motif regardless of fragment size and adding the row sum 4 times to the rightmost side of the 2D vector in order to add fragment size information regardless of fragment end motif.

### Example 4. CNN Model Construction and Training Process

A CNN artificial intelligence model for distinguishing healthy subjects from neuroblastoma cancer patients was trained using the FEMS table or FEMS_Z table two-dimensional vector as an input.

The dataset of Table 2 was used, training dataset was used for model training, the validation dataset was used for hyper-parameter tuning, and the test dataset was used for final model testing.

The basic configuration of the CNN model is shown in FIG. 11. A sigmoid was used as an activation function, 3 convolution layers were used, and 13 10*10 patches were used. For the pooling method, a max mode and a 2×2 patch were used. 4 fully connected layers were used and 454 hidden nodes were included. Finally, the final DPI was calculated using the sigmoid function value.

The hyper-parameter tuning is a process of optimizing the values of various parameters (the number of convolution layers, the number of dense layers, the number of convolution filters, etc.) constituting the CNN model. The hyper-parameter tuning was performed using Bayesian optimization and grid search techniques. When the validation loss started to increase compared to training loss, it was considered that the model was overfitting and model training was stopped.

The performance of several models obtained through hyper-parameter tuning was compared using the validation dataset, the model having the best performance of the validation dataset was determined as the optimal model, and final performance evaluation was performed with the test dataset.

When the FEMS_Z table 2D vector of a random sample was input to the model created through the above process, the probability that the sample is a healthy subject, and the probability that the sample is a neuroblastoma cancer patient were calculated through the softmax function, which is the last layer of the CNN model. Such probability was defined as “deep probability index (DPI)”.

### Example 5. Evaluation of Performance of Constructed Deep-Learning Model Using FEMS_Z Table

### 5-1 Evaluation of Performance (Test)

The performance of the DPI output from the FEMS deep learning model produced in Example 4 and the DPI output by the FEMS_Z deep learning was tested. All samples were divided into training, validation, and test groups. The models were constructed using the training samples, and then the performance of the models constructed using the training samples was evaluated using the samples of the validation and test groups.

TABLE 3

| | Accuracy | | F1-score | | Precision | | AUC | |
|---|---|---|---|---|---|---|---|---|
| | FEMS | FMES_Z | FEMS | FMES_Z | FEMS | FMES_Z | FEMS | FMES_Z |
| Train | 1.000 | 1.000 | 1.000 | 1.000 | 1.000 | 1.000 | 1.000 | 1.000 |
| Validation | 1.000 | 1.000 | 1.000 | 1.000 | 1.000 | 1.000 | 1.000 | 1.000 |
| Test | 0.987 | 1.000 | 0.973 | 1.000 | 0.947 | 1.000 | 1.000 | 1.000 |

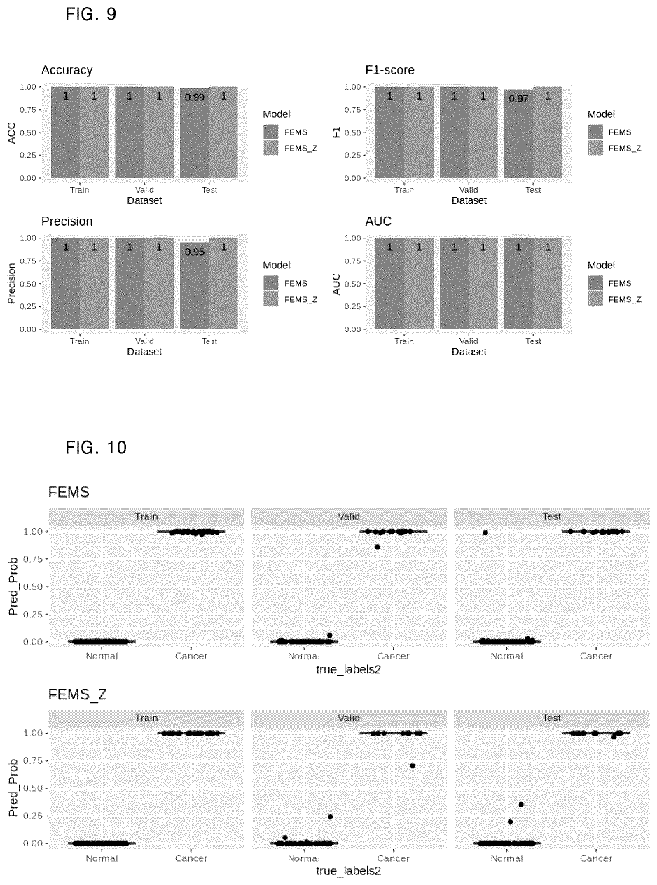

As a result, as can be seen from Table 3 and FIG. 9, accuracy for Train, Valid, and Test groups in the FEMS model was 100%, 100%, and 98.7%, respectively, and accuracy for Train, Valid, and Test groups in the FEMS_Z model was all 100%. Also, the model trained with the FEMS_Z table as an input has excellent performance in terms of F1-score, precision, and AUC.

### 5-2. DPI Distribution

How much the DPI, which is the output value of the deep learning model constructed in Example 5-1, matched the actual patient was determined.

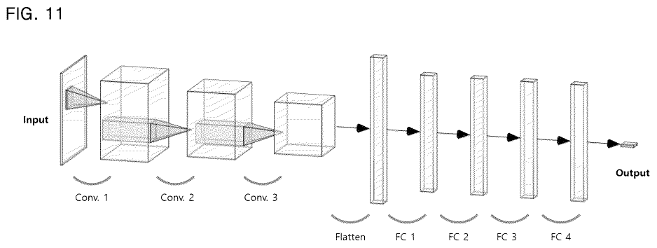

As a result, as can be seen from FIG. 10, the FEMS_Z table learning model was more likely to classify normal as normal and neuroblastoma patients as neuroblastoma patients than the FEMS table learning model.

Although specific configurations of the present invention have been described in detail, those skilled in the art will appreciate that this detailed description is provided as preferred embodiments for illustrative purposes and should not be construed as limiting the scope of the present invention. Therefore, the substantial scope of the present invention is defined by the accompanying filed claims and equivalents thereto.

### INDUSTRIAL APPLICABILITY

The method for diagnosing cancer and predicting cancer types using the cell-free nucleic acid fragment end sequence motif frequency and size according to the present invention exhibits high sensitivity and accuracy in spite of low read coverage because vectorized data is generated and analyzed using an AI algorithm, thus being useful.

### SEQUENCE LISTING FREE TEXT

An electronic file is attached

1. (canceled)

2. A method for diagnosing cancer and predicting a cancer type, the method comprising:
(a) obtaining sequence information from extracted nucleic acids from a biological sample;
(b) aligning the sequence information (reads) with a reference genome database;
(c) acquiring end motif frequencies and sizes of nucleic acid fragments based on the aligned sequence information (reads);
(d) generating vectorized data using the motif frequencies and sizes of nucleic acid fragments;
(e) post-processing the vectorized data;
(f) inputting the post-processed vectorized data into a trained artificial intelligence model, analyzing the data, and comparing an analyzed output value with a cut-off value to determine whether or not cancer develops; and
(g) predicting a cancer type through comparison of the output value.

3. The method according to claim 2, wherein step (a) comprises:
(a-i) extracting nucleic acids from a biological sample;
(a-ii) removing proteins, fats, and other residues from the collected nucleic acids using a salting-out method, a column chromatography method, or a bead method to obtain purified nucleic acids;
(a-iii) producing a single-end sequencing or paired-end sequencing library for the purified nucleic acids or nucleic acids randomly fragmented by an enzymatic digestion, pulverization, or Hydroshear method;
(a-iv) reacting the produced library with a next-generation sequencer; and
(a-v) obtaining sequence information (reads) of the nucleic acids in the next-generation sequencer.

4. The method according to claim 2, wherein the end motif of each nucleic acid fragment in step (c) has a sequence pattern of 2 to 30 bases at both ends of the nucleic acid fragment.

5. The method according to claim 2, wherein the frequency of end motifs of the nucleic acid fragments in step (c) corresponds to the number of motifs detected in all the nucleic acid fragments.

6. The method according to claim 2, wherein the size of each nucleic acid fragment in step (c) corresponds to the number of bases from the 5′ end to the 3′ end of the nucleic acid fragment.

7. The method according to claim 2, wherein the vectorized data in step (d) is expressed by a type of the end motif of the nucleic acid fragment plotted on an X-axis and a size of the nucleic acid fragment plotted on a Y-axis.

8. The method according to claim 2, wherein step (e) is performed by a method including the following steps:
(e-i) calculating the mean and standard deviation of the frequency of the end motifs of the nucleic acid fragment and size of the nucleic acid fragment in a normal group;
(e-ii) subtracting the mean of the frequency for the end sequence motif type of each nucleic acid fragment and the size of nucleic acid fragment in the normal group from the frequency for the end sequence motif type of each nucleic acid fragment and the size of nucleic acid fragment in the sample, and dividing the result by the standard deviation of the frequency for each type of motif and size of the nucleic acid fragment to perform Z standardization and thereby obtain a Z-standardized value; and
(e-iii) correcting the Z-normalized value derived in step (e-ii) with a cut-off value when the Z-normalized value exceeds a cut-off range.

9. The method according to claim 8, wherein the cut-off range is −5 to 5 and the cut-off value is −5 or 5.

10. The method according to claim 7, wherein the vectorized data further comprises a sum of frequencies for end motifs of nucleic acid fragments and a sum of frequencies for sizes of nucleic acid fragments.

11. The method according to claim 2, wherein the artificial intelligence model in step (f) is trained to distinguish between vectorized data of a healthy subject and vectorized data of a cancer patient.

12. The method according to claim 11, wherein the artificial intelligence model is selected from the group consisting of a convolutional neural network (CNN), a deep neural network (DNN), and a recurrent neural network (RNN).

13. The method according to claim 12, wherein, when the artificial intelligence model is a CNN, a loss function for performing binary classification is represented by Equation 1 below and a loss function for performing multi-class classification is represented by Equation 2 below:
$$Binary classification$$
$$loss ( model ( x ) , y ) = - \frac{1}{n} [ {{\sum }_{i = 1}}^{n} ( {y}_{i} log ( model ( {x}_{i} ) ) + ( 1 - {y}_{i} ) log ( 1 - model ( {x}_{i} ) ) ) ] \qquad Equation 1$$
Model (xᵢ)=Artificial intelligence model output in response to ith input
y=Actual label value
n=Number of input data
$$Multi - class classification$$
$$loss ( model ( x ) , y ) = - \frac{1}{n} {{\sum }_{i = 1}}^{n} ( {\sum }_{j = 1}^{c} ( {y}_{ij} {log ( model ( {x}_{i} ) )}_{j} ) \qquad Equation 2$$
Model (xᵢ)ⱼ=j^{th} artificial intelligence model output in response to i^{th} input
y=Actual label value
n=Number of input data
c=Number of classes

14. The method according to claim 2, wherein the output value resulting from analysis of the input vectorized data by the artificial intelligence model in step (f) is a deep probability index (DPI).

15. The method according to claim 2, wherein the cut-off value of step (f) is 0.5 and a determination is made that cancer has developed when the output value is 0.5 or more.

16. The method according to claim 2, wherein step (g) of predicting the cancer type through comparison of the output value comprises determining a type of cancer showing the highest DPI among the calculated DPIs for respective cancer types as the cancer type of the sample.

17. A device for diagnosing cancer and predicting a cancer type, the device comprising:
a decoder configured to extract nucleic acids from a biological sample and decode sequence information;
an aligner configured to align the decoded sequences with a reference genome database;
a nucleic acid fragment analyzer configured to acquire end motif frequencies and sizes of nucleic acid fragments based on the aligned sequences;
a data generator configured to generate vectorized data using the end motif frequencies and sizes of nucleic acid fragments and then perform post-processing;
a cancer diagnostic unit configured to input the post-processed vectorized data into a trained artificial intelligence model, analyze the data, compare a resulting output value with a cut-off value, and thereby determine whether or not cancer develops; and
a cancer type predictor configured to analyze the output value and thereby predict the cancer type.

18. A computer-readable storage medium for diagnosing cancer and predicting a cancer type including an instruction configured to be executed by a processor for diagnosing cancer and predicting a cancer type through the following steps comprising:
(a) extracting nucleic acids from a biological sample to obtain sequence information;
(b) aligning the sequence information (reads) with a reference genome database;
(c) acquiring end motif frequencies and sizes of nucleic acid fragments based on the aligned sequence information (reads);
(d) generating vectorized data using the motif frequencies and sizes of nucleic acid fragments;
(e) post-processing the generated vectorized data;
(f) inputting the post-processed data to a trained artificial intelligence model, analyzing the data, and comparing an analyzed output value with a cut-off value to determine whether or not cancer develops; and
(g) predicting a cancer type through comparison of the output value.
